(19) United States
(12) Patent Application Publication
(10) Pub. No.: US 2015/0088041 A1
(43) Pub. Date: Mar. 26, 2015

(54) HEAD COVER FOR MASSAGE CHAIR

(71) Applicant: AFAR USA LLC, Fullerton, CA (US)

(72) Inventors: Ming-Kuo Tseng, Taichung City (TW); Gin-Feng Tseng, Taichung City (TW)

(73) Assignee: AFAR USA LLC, Fullerton, CA (US)

(21) Appl. No.: 14/033,416

(22) Filed: Sep. 20, 2013

(51) Int. Cl. A61B 18/18

(52) U.S. Cl. CPC A61B 18/18; A61B 2018/1807
USPC 601/18

(57) ABSTRACT

A massage chair with a head cover is disclosed. The massage chair has an inner hat body, an outer shell and a lighting element at a front end of the outer shell. The lighting element can generate four frequency bands α, β, θ, δ, and the combination thereof, so when the user is using the massage chair, the user can not only the inner hat body and the outer shell can prevent the user from being disturbed, but also the lighting element can generate light waves to the user's eyes, so the user's optic can receive the light waves and convert them to signals transmitted to the brain to achieve the results of spiritual guidance.

### FIELD OF THE INVENTION

The present invention relates to a massage chair, and more particularly to a massage chair with a head cover.

### BACKGROUND OF THE INVENTION

Nowadays, people are busy at work and lack of exercise, or having a fixed position for a long time, so the body tends to be painful in some portions and all kinds of massaging equipment emerge on the market. For current massage chairs, their features cover the entire spectrum, from head to toe, and even the wrist arm. Although the price is not low, there are more and more consumers purchase the massage chairs because they care more about their health and quality of life. However, current massage chairs are designed primarily for pressing or hammering muscles to achieve the purpose of relaxation, so when the massaging is in progress, the user's brainwave and eyes are vulnerable to external influence such as light, sound, etc., and it is difficult for the user to be truly relaxed.

### SUMMARY OF THE INVENTION

The technical problem the present invention wants to solve is that current massage chairs are designed primarily for pressing or hammering muscles to achieve the purpose of relaxation, so when the massaging is in progress, the user's brainwave and eyes are vulnerable to external influence such as light, sound, etc., and it is difficult for the user to be truly relaxed.

The present invention provides a massage chair with a head cover may include a massage chair, an inner hat body, an outer shell and a lighting element. A rail is located at both sides of a head portion of the massage chair, and the inner hat body connects to the head portion. A restricting guiding slot is formed at two inner walls of the inner hat body corresponding to the rails, and a conjugating hole is formed at both sides of the outer surface of the inner hat body. The conjugating hole is further be used as an axle center for a U-shaped unit on both sides. Both sides of the outer shell have a through hole corresponding to the conjugating hole of the inner hat body, and there are two locking units. A fan-shaped connecting piece is disposed at both inner sides of the outer shell and both connecting pieces are disposed between the inner hat body and the outer shell. The connecting piece has a connecting hole corresponding to the conjugating hole and the through hole, and a bearing unit is disposed at both side of the outer portion of the inner hat body, and an anti-slippery unit is disposed at both sides of the bearing unit. Also, two restricting protruding units are disposed near the anti-slippery units using the connecting holes as axel centers, and an engaging hole is formed at a front end of the outer shell. The lighting element has an inner shell body, an outer shell body and a lighting body. The inner shell body and outer shell body engages with each other at an inner and outer portion of the engaging hole of the outer shell. An O-shaped ring is disposed at the inner shell body that is at an inner portion of the engaging hole, and a pivot portion is disposed at the outer shell body that is outside the engaging hole. The lighting body is disposed between the inner and outer shell bodies, and the lighting body has a connecting post corresponding to the pivot portion on both sides of the outer shell body, so the lighting body can be disposed inside the outer shell body. The lighting body is controlled by a controller to generate light waves, frequency of which is one of four frequency bands α, β, θ, δ and the combination thereof.

The present invention is advantageous because the massage chair with the head portion has the inner hat body, the outer shell and the lighting element at the front end of the outer shell. The lighting element can generate four frequency bands α, β, θ, δ or the combination thereof, so when the user is using the massage chair, the user can not only the inner hat body and the outer shell can prevent the user from being disturbed, but also the lighting element can generate light waves to the user's eyes, so the user's optic can receive the light waves and convert them to signals transmitted to the brain to achieve the results of spiritual guidance.

### BRIEF DESCRIPTION OF THE DRAWINGS

FIG. 1 illustrates a three-dimensional view of the present invention.

FIG. 2 illustrates an exploded view of the present invention.

FIG. 3 illustrates a partial enlarged sectional view of the present invention.

FIG. 4 illustrates a schematic view of the enlarged outer shell when lifted in the present invention.

FIG. 5 illustrates a schematic view of the enlarged outer shell when covered down in the present invention.

FIG. 6 illustrates a schematic view of the lighting element when it emits light.

FIG. 7 illustrates a schematic view of the lighting element when it is adjusted.

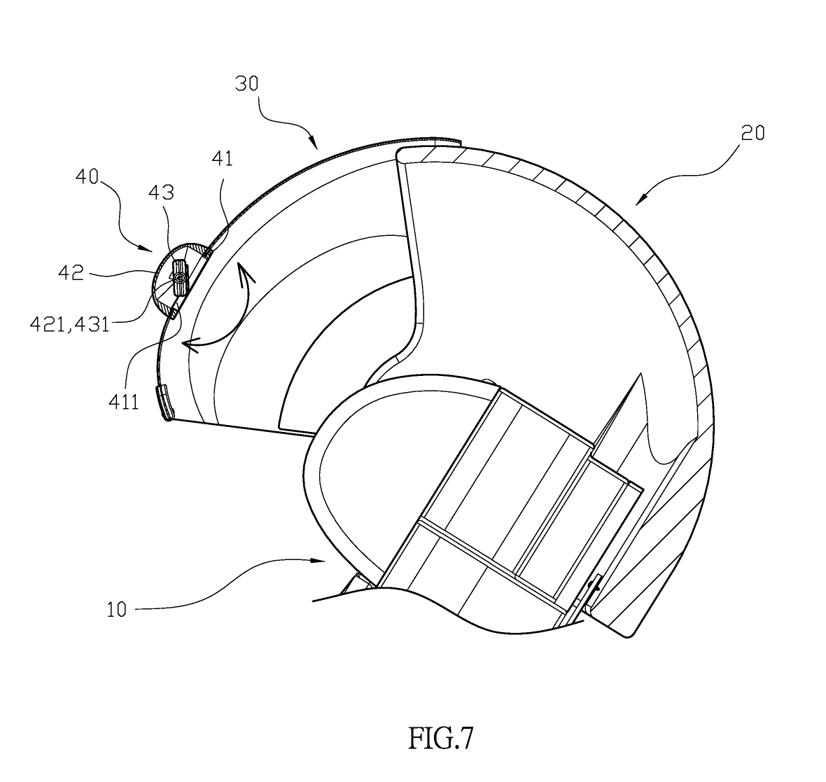

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as following:

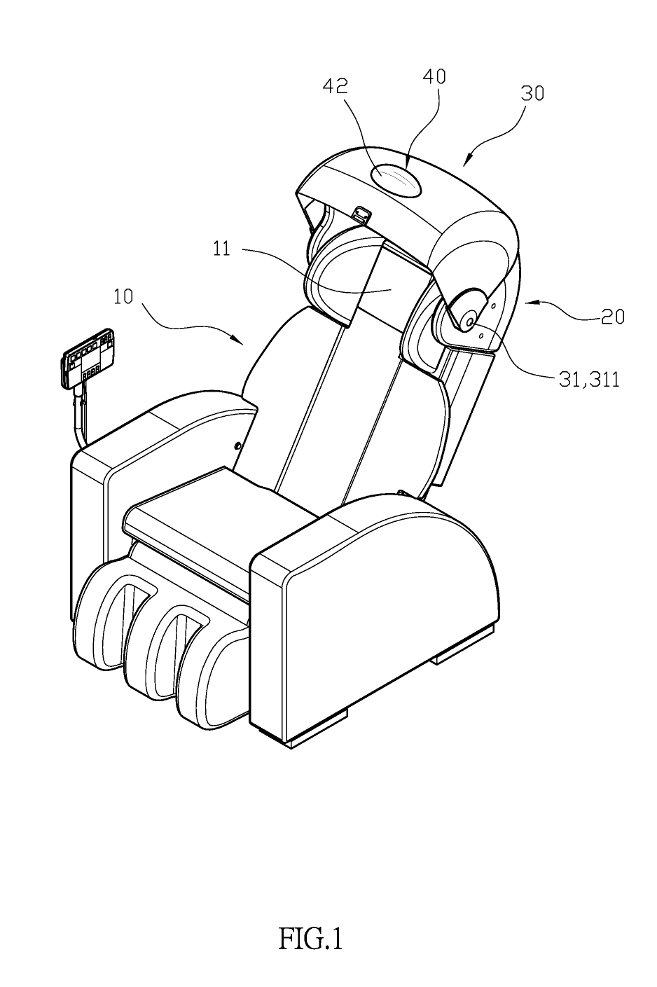

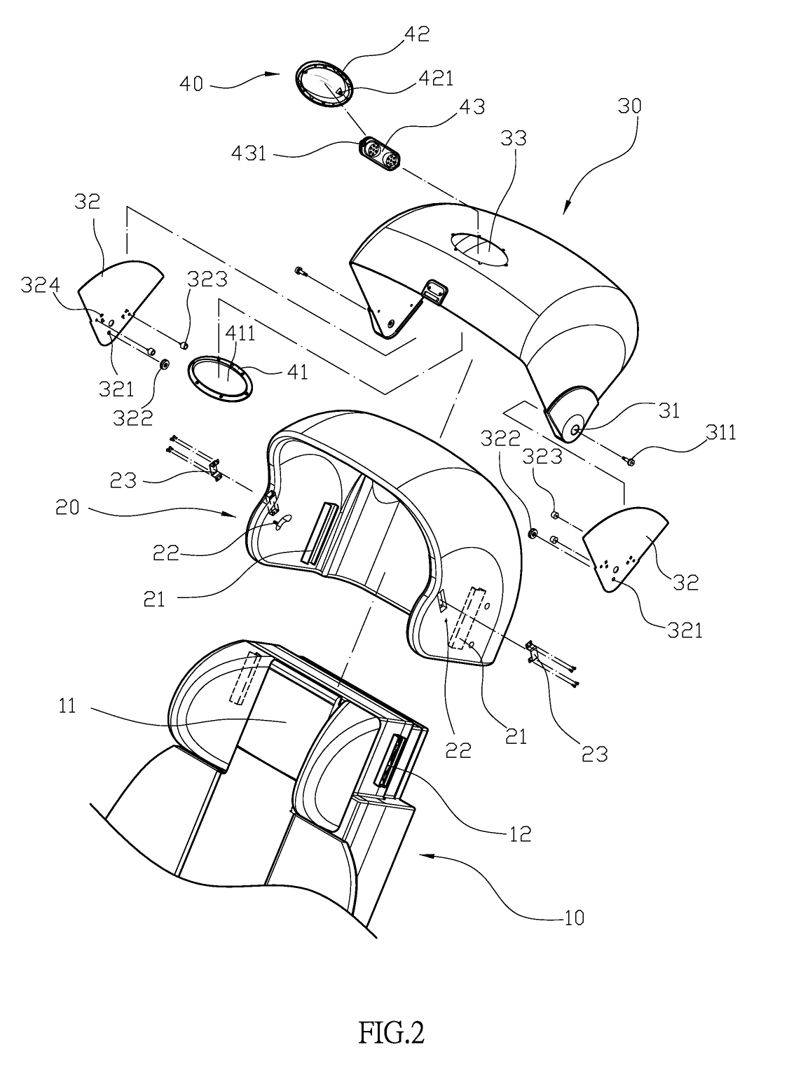

Referring to FIGS. 1 and 2, a massage chair with a head cover may include a massage chair 10, an inner hat body 20, an outer shell 30 and a lighting element 40. A rail 12 is located at both sides of a head portion 11 of the massage chair 10, and the inner hat body 20 connects to the head portion 11. A restricting guiding slot 21 is formed at two inner walls of the inner hat body 20 corresponding to the rails 12, and a conjugating hole 22 is formed at both sides of the outer surface of the inner hat body 20. The conjugating hole 22 is further be used as an axle center for a U-shaped unit 23 on both sides. Both sides of the outer shell 30 have a through hole 31 corresponding to the conjugating hole 22 of the inner hat body 20, and there are two locking units 311. A fan-shaped connecting piece 32 is disposed at both inner sides of the outer shell 30 and both connecting pieces 32 are disposed between the inner hat body 20 and the outer shell 30. The connecting piece 32 has a connecting hole 321 corresponding to the conjugating hole 22 and the through hole 31, and a bearing unit 322 is disposed at both side of the outer portion of the inner hat body 20, and an anti-slippery unit 323 is disposed at both sides of the bearing unit 322. Also, two restricting protruding units 324 are disposed near the anti-slippery units 323 using the connecting holes 321 as axel centers, and an engaging hole 33 is formed at a front end of the outer shell 30. The lighting element 40 has an inner shell body 41, an outer shell body 42 and a lighting body 43. The inner shell body 41 and outer shell body 42 engages with each other at an inner and outer portion of the engaging hole 33 of the outer shell 30. An O-shaped ring 411 is disposed at the inner shell body 41 that is at an inner portion of the engaging hole 33, and a pivot portion 421 is disposed at the outer shell body 42 that is outside the engaging hole 33. The lighting body 43 is disposed between the inner and outer shell bodies (41, 42), and the lighting body 43 has a connecting post 431 corresponding to the pivot portion 421 on both sides of the outer shell body 42, so the lighting body 43 can be disposed inside the outer shell body 42. The lighting body 43 is controlled by a controller to generate light waves, frequency of which is one of four frequency bands α, β, θ, δ and the combination thereof.

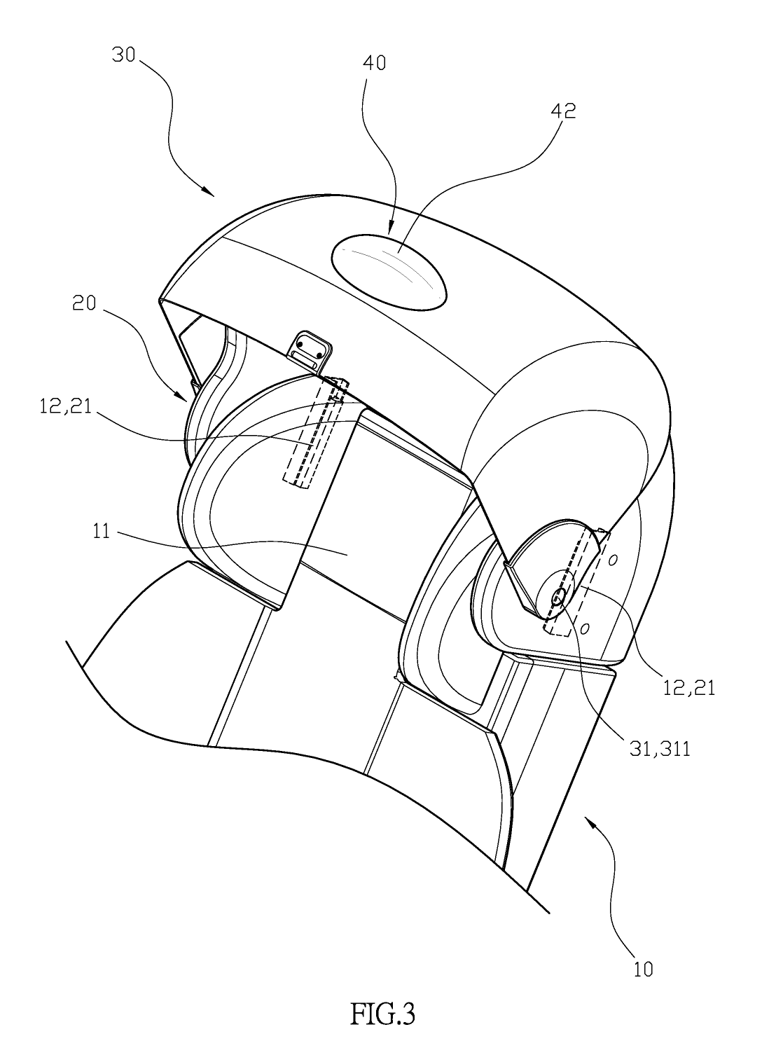

Referring to FIGS. 2 to 3 for the structure, the outer shell 30 is disposed outside the inner hat body 20 and the connecting piece 32 is between the inner hat body 20 and the outer shell 30. Outer surface of the connecting pieces 32 has an anti-slippery unit 323 corresponding to the inner hat body 20, and the other end of the anti-slippery unit 323 is connected to the outer shell 30 through the connecting piece 32, so that the connecting hole 321 of the connecting piece 32 can align with the conjugating hole 22 of the inner hat body 20 and the through hole 31 of the outer shell 30. Also, the bearing unit 322 is disposed between the connecting piece 32 and the inner hat body 20, and the locking unit 311 is inserted into the through hole 31 of the outer shell 30, the connecting piece 32 and the bearing unit 322, and is tightened at the conjugating hole 22. Thus, the outer shell 30 can be disposed outside the inner hat body 20 through the connecting piece 32, and the outer shell 30 and the connecting piece 32 can be moved around the locking unit 311. Furthermore, two restricting protruding units 324 using the connecting holes 321 as axel centers are used to restrict the movement of the outer shell 30. The engaging hole 33 of the outer shell 30 has the lighting element 40, which is formed by engaging the pivot portion 421 of the outer shell body 42 and the connecting post 431 of the lighting body 43, so the lighting body 43 can be adjusted at the pivot portion 421 through the connecting post 431. The inner shell body 41 and outer shell body 42 are further secured at the engaging holes 33 of the outer shell 30. According to the structure described above, the inner hat body 20, outer shell 30 and the lighting element 40 are assembled, and the restricting guiding slot 21 of the inner hat body 20 is disposed in the rail 12 on both sides of the massage chair 10 to complete the entire assembly process.

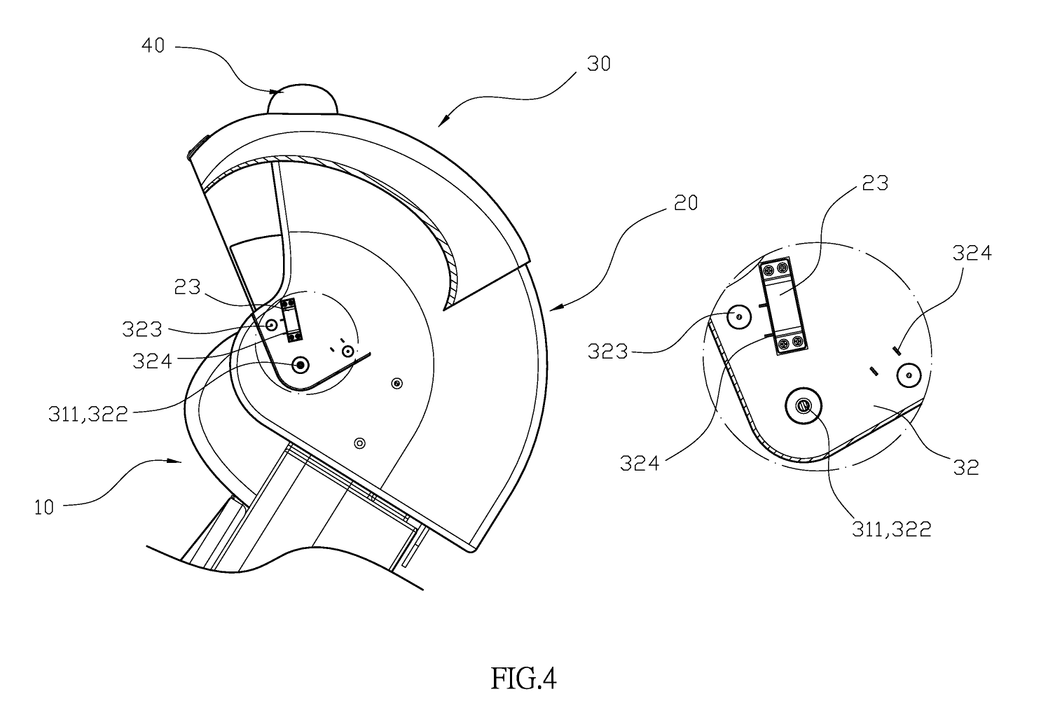

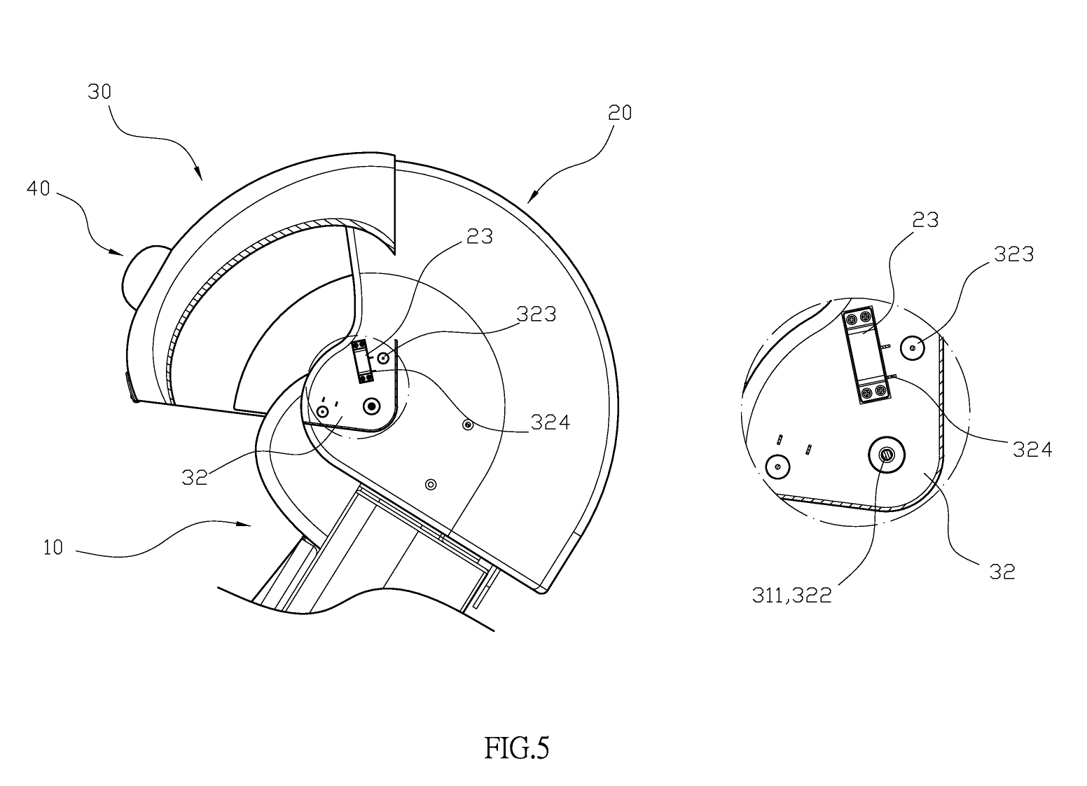

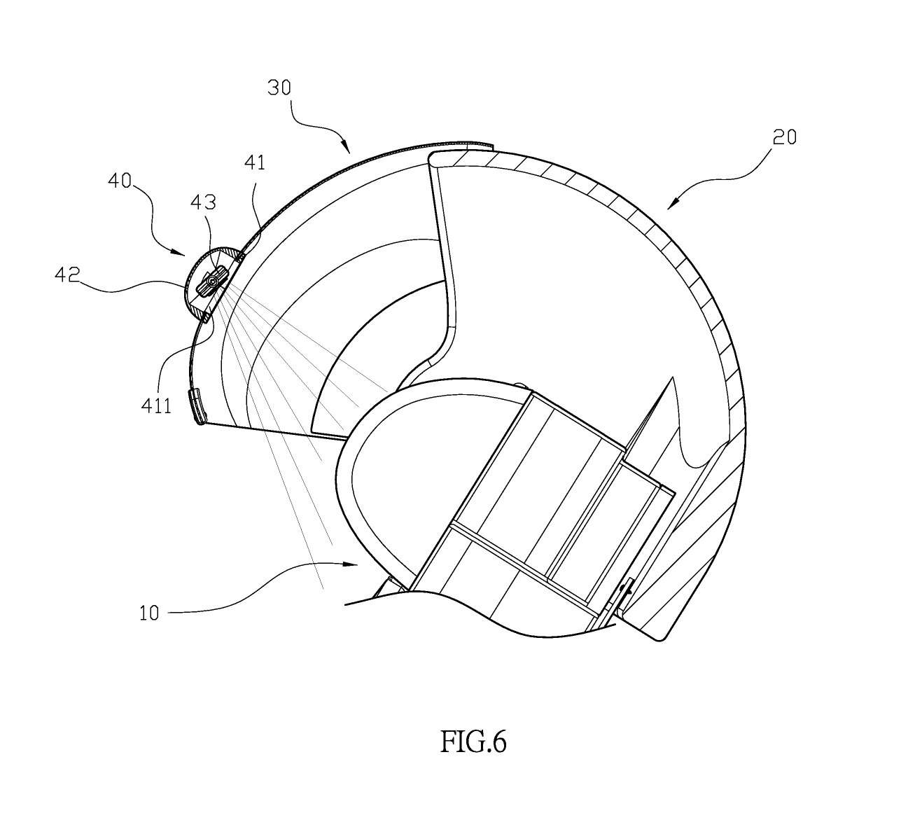

Referring to FIGS. 4 to 6, when the massage chair is in use, the user's head is disposed at the head portion 11 of the massage chair 10, and is covered by the inner hat body 20 that has a lifted outer shell 30, and the restricting protruding unit 324 is disposed against the U-shaped unit 23 of the inner hat body 20 to form a secured positioning. Thus, when the user sits on the massage chair 10 and the user's head is on the head portion 11, the outer shell 30 can be moved down through the locking unit 311, and the restricting protruding unit 324 is disposed against the U-shaped unit 23 of the inner hat body 20 to form a secured positioning, so the movement of the outer shell 30 can be restricted when it is moved down. Furthermore, the anti-slippery unit 323 on the surface of the connecting piece 32 is disposed against the surface of the inner hat body 20 to prevent the outer shell 30 from moving down. When the outer shell 30 covers down, the lighting element 40 faces the user's eyes and the user can control the lighting body 43 to emit one of the four frequency bands α, β, θ, δ. When the user needs to rest, the frequency band can be adjusted to α, and when the user has to think about something, frequency β may be preferable. And when the user needs to take a nap, frequency θ may be preferable while frequency δ is good for people who need deep sleep. In another embodiment, the frequency can be a combination of two, three or four frequencies, so the frequency can be transmitted to signals from user's optic to brain to generate healing effects.

Also, the lighting element 40 on the outer shell 30 can be adjusted according to the user's posture. When the user is sitting on the massage chair, the lighting body 43 of the lighting element 40 can be adjusted, so that the lighting body 43 can be more focused on the user's eyes to enhance the healing effect.

According to the embodiments described above, the present invention is advantageous because the massage chair 10 with the head portion 11 has the inner hat body 20, the outer shell 30 and the lighting element 40 at the front end of the outer shell 30. The lighting element 40 can generate four frequency bands α, β, θ, δ or the combination thereof, so when the user is using the massage chair, the user can not only the inner hat body 20 and the outer shell 30 can prevent the user from being disturbed, but also the lighting element 40 can generate light waves to the user's eyes, so the user's optic can receive the light waves and convert them to signals transmitted to the brain to achieve the results of spiritual guidance.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A massage chair with a head cover comprising:
a massage chair, having a rail located at both sides of a head portion of the massage chair;
an inner hat body connected to the head portion, a restricting guiding slot formed at two inner walls of the inner hat body corresponding to the rails, a conjugating hole formed at both sides of outer surface of the inner hat body, wherein said conjugating hole is further used as an axle center for a U-shaped unit on both sides of the inner hat body;
an outer shell, having a through hole on both sides thereof corresponding to the conjugating hole of the inner hat body with two locking units, a fan-shaped connecting piece disposed at both inner sides of the outer shell and both connecting pieces are disposed between the inner hat body and the outer shell, wherein the connecting piece has a connecting hole corresponding to the conjugating hole and the through hole, and a bearing unit is disposed at both side of the outer portion of the inner hat body, and an anti-slippery unit is disposed at both sides of the bearing unit, wherein two restricting protruding units are disposed near the anti-slippery units using the connecting holes as axel centers, and an engaging hole is formed at a front end of the outer shell; and
a lighting element, having an inner shell body, an outer shell body and a lighting body, wherein the inner shell body and outer shell body engages with each other at an inner and outer portion of the engaging hole of the outer shell, and an O-shaped ring is disposed at the inner shell body that is at an inner portion of the engaging hole, and a pivot portion is disposed at the outer shell body that is outside the engaging hole, wherein the lighting body is disposed between the inner and outer shell bodies, and the lighting body has a connecting post corresponding to the pivot portion on both sides of the outer shell body, so the lighting body is disposed inside the outer shell body.

2. The massage chair with a head cover of claim 1, wherein a controller of the lighting body is used to emit light waves, and the frequencies of the light waves include α, β, θ, δ, and the combination thereof.
